(19) United States
(12) Patent Application Publication
(10) Pub. No.: US 2007/0255402 A1
(43) Pub. Date: Nov. 1, 2007

(54) WOUND CLOSURE METHOD

(76) Inventors: Jacob J. Moore, Harlingen, TX (US); David H. Pashley, Augusta, GA (US); Balamurali K. Ambati, (US)

Correspondence Address: HAYES SOLOWAY P.C., 3450 E. SUNRISE DRIVE, SUITE 140, TUCSON, AZ, 85718, US

(21) Appl. No.: 11/743,094

(22) Filed: May 1, 2007

Related U.S. Application Data
(60) Provisional application No. 60/746,099, filed on May 1, 2006.

(51) Int. Cl. A61F 2/14

(52) U.S. Cl. 623/5.11; 623/23.72; 606/4; 523/118; 424/78.02

(57) ABSTRACT

A method for convenient, controlled wound closure of an eye, the skin, internal organs and other soft tissue which comprises administering to the wound a sterile, body compatible photopolymerizable acrylate or methacrylate adhesive, and exposing the applied adhesive to light.

### CROSS REFERENCE TO RELATED APPLICATION

This application claims priority to U.S. Provisional Application Ser. No. 60/746,099, filed May 1, 2006, which is entirely incorporated herein by reference.

### FIELD OF THE INVENTION

The present invention relates to improvements in surgical processes. The invention has particular utility in connection with corrective eye surgeries such as corneal tissue transplant, and with soft tissue surgery where suture placement is either undesirable due to scarring or impractical due to technical limitations. The invention will be described in connection with corrective eye surgery, although other utilities are contemplated.

### BACKGROUND ON THE INVENTION

A common form of corrective eye surgery is keratoplasty, the transplanting of corneal tissue from a donor to a patient with corneal problems. Advances in the field of keratoplasty have considerably increased the rate of success in these operations. However, this success rate usually relates to the attaining of a clear cornea. There remains a problem with these operations in that post-operative astigmatism following the corneal graft occurs in a large number of cases, and this can severely limit the visual acuity of the patient.

The use of sutures necessarily distorts a wound creating regular or irregular astigmatism in the cornea that may not be corrected with eyeglasses Attempts at controlling this distortion have largely been limited to the development of different suturing techniques. These techniques have included the use of different sized non-reactive Nylon sutures, the use of continuous running Nylon sutures, sometimes in combinations with interrupted corneal sutures, and other methods. Despite all of these attempts at reducing astigmatism, the results have fallen far short of ideal. Recent studies have shown that astigmatism following suture removal has been largely unaffected by these various suturing techniques. The methods used thus far have not been successful in reducing final astigmatism following corneal transplant and suture removal.

Moreover, use of sutures in the skin also leads to distortion of the wound, with increasing amounts of scarring that may be elevated, depressed or abnormally pigmented. The use of different non-reactive suture materials such as Nylon and polypropylene, and different suture techniques in the skin such as absorbable and non-absorbable subcuticular sutures, stainless-steel staples and other methods have been developed to provide either decreased scarring or ease of wound closure respectively. Again wound closure methods are far from ideal.

Tissue adhesives such as cyanocrylates have been used in dentistry, plastic surgery, and as an off-label use in corrective eye surgery as a conservative measure in the setting of corneal ulcers. These ulcers sometimes develop on the cornea due to infectious or autoimmune disease that are both painful and dangerous if they enlarge. These are often treated with cyanoacrylate adhesives to prevent progressive corneal thinning and perforation. Commercially available cyanoacrylates such as n-octyl cyanoacrylate (Dermabond™) are used in the setting of cosmetic plastic surgery to provide for wound closure with ease of application and reduced scarring. However, cyanoacrylate adhesives polymerize spontaneously as soon as they touch the skin or moist surface of the eye. This causes uneven spreading of such films and produces a rough irregular surface that can be irritating. Cyanoacrylate adhesives also are difficult to control and may spread beyond the area intended. Therefore cyanoacrylate adhesives are not ideal for wound closure in the eye or of soft tissue such as skin.

### SUMMARY OF THE INVENTION

The above and other disadvantages of the prior art are overcome by the present invention, which relates to a simple, rapid method of wound closure using certain body compatible, sterile photopolymerizable adhesives.

### DETAILED DESCRIPTION OF PREFERRED EMBODIMENTS

Dental adhesive technology has advanced over the last two decades with the development of self-etching primers that require no rinsing. These solutions are applied with very small (ca. 1 mm diameter) disposable microsponge/microbrushes or with a small bore cannula on a syringe. Under an operating microscope, light-cured dental adhesives can be applied with great control and then cured with a blue-light (450-490 nm) in 10 sec. A particular feature and advantage of such light-cured adhesives is that they do not polymerize in the presence of moisture, but only after the blue-light is directed to the wound, e.g. with a fiberoptic light guide. They also are transparent in visible light, once cured. This makes light-cured dental adhesives ideal for use in moist environments such as the eye, skin and internal organs. While the use of methacrylate base adhesives in dental technology is well known, there have been no previous public reports of using light-cured methacrylate adhesives in soft tissue such as the cornea, skin, or internal organs.

Thus, this invention in one aspect relates to a method of wound closure by controlled application of selected light-cured or photopolymerizable adhesives in place of conventional suturing for repairing wounds of the cornea, skin or internal organs. The light-cured adhesives may be applied by first applying a solvated primer solution, followed by application of a non-solvated adhesive and exposure to UV light for sufficient time to cure the applied layer. Alternatively, a non-solvated photopolymerizable adhesive or a solvated photopolymerizable adhesive blend may be applied in a single step.

Particularly useful in the practice of the present invention are UV light photopolymerizable acrylic and methacrylic adhesives that have been developed for use in the dental industry and are available from a variety of manufacturers including 3M and Kuraray Medical (Japan). However, other photopolymerizable adhesives which may be UV light or visible light polymerizable may be employed.

Photopolymerizable acrylic and methacrylic adhesives that have been developed for use in the dental industry adhesives are typically comprised of a solution of MDP (methacryloyloxydecyl phosphate), HEMA (hydroxyethylmethacrylate), dimethyacrylate monomer, camphorquinone, and water. Most dental resins include hydrophilic monomethacrylates such as hydroxyethyl methacrylate (HEMA) because it is miscible with water and water-saturated surfaces. These comonomer blends usually include dimethacrylates such as triethyleneglycol dimethacrylate (TEGDMA) or 4,2-hydroxy-3-methacryloyloxypropoxyphenyl propane (BisGMA) to cross-link the polymer network thereby increasing its strength. However, a disadvantage of including dimethacrylates such as TEGDMA and BisGMA is that they are more hydrophobic and poorly miscible with water. Indeed, they can form multiple phases if exposed to too much water, leading to poor film formation and weak adhesion.

To improve surface wetting and film spreading, comonomer blends are usually solvated with acetone, ethanol or water, or mixtures of these solvents. This lowers the comonomer concentration and requires evaporation of the solvents prior to photopolymerization to increase the comonomer concentration enough for good radical propagation between growing polymer chains.

Recently, advances have been developed to homogenize hydrophobic resins into hydrophilic resins to reduce or eliminate the phase changes seen with mixtures of hydrophilic and hydrophobic comonomers are applied to water-rich surfaces (Spencer and Wang, 2002). The use of monomers with surfactant properties, coupled with ultrasonication can create stable nanodispersions of hydrophobic monomers in hydrophilic monomer/solvent blends.

The advantage of using hydrophilic monomers to create adhesive polymers is that they attract and absorb water. The water hydrogen bonds with the hydrophilic polar groups on the polymers thereby causing swelling of the film and lowers its mechanical properties (Yiu et al., 2004). Hydrophilic polymers also have high solubilities that slowly promotes debonding of the adhesive film from the skin or mucous membrane. The rate of these processes can be controlled by changes in the concentration of hydrophilic monomers. Thus, the duration of adhesion can be modified to some degree to provide different healing periods prior to the spontaneous loss of the adhesive film.

Particularly preferred in the practice of the invention are commercially available all-in-one dental adhesives such as Clearfil tri-S Bond (Kuraray Medical, Tokyo, Japan) or Xeno III (Caulk/Dentsply, Milford, Del., USA). Clearfil tri-S Bond is comprised of a solution of 2-hydroxyethyl methacrylate, bisphenol a diglycidylmethacrylate, silanated colloidal silica, 10-methacryloyloxydecyl dihydrogen phosphate, d,1-camphorquinone, ethyl alcohol, and water.

Further details and advantages of the invention will be seen from the following examples which are intended to be purely exemplary of the intention and are not intended to limit the scope of what the inventors regard as their invention. Unless stated otherwise, parts are parts by weight. Temperature is in ° C. or is at room temperature, and pressure is at or near atmospheric.

### EXAMPLE 1

Fifteen fresh excised pig eyes were obtained from the local abattoir and kept at 4° C. until use. They were cleaned of extraneous tissue and then the sclera, including the optic nerve and episcleral vessels were sealed with a viscous cyanoacrylate cement (Zapit, Dental Ventures of American, Corona, Calif.). The anterior chamber of the eye was then cannulated with a 23 ga. needle connected by polyethylene tubing to an automated fluid flow measuring device (FLODEC, DeMarco Engineering, Geneva, Switzerland) to track the movement of a tiny air bubble through a calibrated glass capillary. The entire system including the anterior chamber was filled with sterile phosphate buffered saline (PBS). Fluid egress from the eye, as a function of intraocular pressure was measured for 5 control eyes with 5 mm incisions closed with 4-5 simple interrupted 10-0 nylon sutures, and 5 experimental eyes with 5 mm incisions closed with a commercially available photopolymerizable adhesive containing hydroxylethyl methacrylate (Clearfil SE Bond dental adhesive available from Kurare, Japan) To test the leakage of control pig eyes, the PBS-filled FLODEC fluid reservoir was raised from zero cm H₂O hydrostatic pressure to 20 cm H₂O (normal intraocular pressure) to 40 cm, 60, 80, 100 and 120 cm H₂O. At each increase in pressure, the globe increased its volume due to its relatively low compliance, but after reaching a steady-state, the “leak rate” of an intact cornea was only 2.3 μ.L/min at an intraocular pressure of 30 cm H₂O. After making a penetrating 5 mm incision through the cornea and closing it with 4-5 interrupted sutures, the leak rate increased significantly (p <0.05) to 12.7 μL/min (Table 1). When experimental eyes with 5 mm incisions were closed with a photopolymerizable HEMA-containing adhesive, subsequent leak tests revealed a leak rate of only 3.2 μL/min, a value that was not statistically different (p=0.56) from nonincised control corneas.

TABLE 1
Fluid leakage across cornea

| | |
|---|---|
| Intact cornea | 2.3 ± 0.9 μL/min^{a} |
| Sutured cornea | 12.7 ± 3.1 μL/min^{b} |
| Resin sealed cornea | 3.2 ± 1.1 μL/min^{a} |

Values are mean ± SD (n = 5). Flow rate measured at 30 cm H₂O. Groups identified by different superscript letters are significantly different (p < 0.05).

These repaired corneal wounds were then subjected to step-wise increases in intraocular pressure until the leak rates suddenly increased to rates>100 μL/min. This occurred at an intraocular pressure of 73.3 cm H₂O for sutured corneas, but required 140 cm H₂O in the wounds closed with a photopolymerizable adhesive as above described.

### EXAMPLE 2

In another experiment, after making 5 mm penetrating wounds of the cornea and then closing them with either sutures or photopolymerizable adhesive, 2×0.5 cm strips of cornea containing the repaired wounds in the middle, were excised and placed in PBS to permit testing of the tensile strength of the repaired wounds. They were then attached to a testing jig designed to apply tensile loading from a universal testing machine at a rate of 12 mm/min as specified by ASTM standard for viscoelastic materials (ASTM D412, 2002).

The controls were 2.0×0.5 cm long strips on unincised corneal tissue that were pulled in tension to yield the ultimate tensile strength (i.e. cohesive strength) of the tissue. The control corneal strength was 0.57±0.15 N/mm² (mean±SD, n=5). The immediate tensile strength of corneal wounds closed with 4-5 simple interrupted 10-0 nylon sutures was 0.36±0.14 N/mm². The tensile strength of corneal wounds closed with the proprietary photopolymerizable adhesive was 0.37±0.10 N/mm² (n=5). These values were not statistically different (p>0.05, Table 2).

TABLE 2
Strength wound closure (N/mm²)

| | |
|---|---|
| 4 Sutures | 0.36 ± 0.14 (5)^{a} |
| Adhesive | 0.37 ± 0.10 (5)^{a} |
| No wound | 0.55 ± 0.15 (5)^{a} |

Values are mean ± SD (n). Groups identified by the same superscript letter are not significantly different (p > 0.05).

Employing a light-cured adhesive in place of conventional sutures to close corneal wounds has other advantages. Placing 4-5 simple interrupted sutures of 10-0 nylon using an operating microscope under ideal lighting typically takes at least 20 min. Applying the adhesive and light-curing takes about 1 min. The use of sutures leaves the wound margins puckered and can induce irregular corneal astigmatism, which may or may not be corrected with glasses. Adhesives do not distort the corneal wound architecture and do not produce the irregular astigmatism encountered with sutures.

Thus, the use of light-cured dental resins to close corneal wounds provide significant advantages over conventional suturing. Moreover, the invention also may be used for closing wounds in other nonstressed regions such as the eyelids, skin, etc. Furthermore, this invention also may be used in situations where suture placement might prove to be too technically difficult, e.g. during endoscopic or other minimally invasive surgical procedures. This method of wound closure also may be useful in anxious, needle-phobic patients, as well as in cosmetic plastic surgery or for wound repair in the field, e.g. in military combat.

The adhesives may be supplied non-solvated with a separate primer solution, or in a single bottle as a solvated blend. Also, in a preferred embodiment of the invention, the adhesive is provided as part of a sterile single dose unit in a surgery kit that also contains disposable microsponges, syringes and cannulae or other devices for application in addition to disposable wells for dispensing.

Various modifications and variations can be made in the present invention without departing from the scope or spirit of the invention. It is intended that the specification and examples be considered as exemplary only, with the true scope and spirit of the invention being indicated by the following claims:

We claim:

1. A method for convenient, controlled wound closure of an eye, the skin, internal organs and other soft tissue which comprises administering to the wound a sterile, body compatible photopolymerizable acrylate or methacrylate adhesive, and exposing the applied adhesive to light.

2. The method of claim 1, wherein the wound comprises a corneal wound.

3. The method of claim 2, wherein the corneal wound comprises a corneal transplant.

4. The method of claim 1, wherein the wound comprises a skin wound.

5. The method of claim 2, wherein the wound comprises a traumatic wound.

6. The method of claim 4, wherein the wound comprises a wound in cosmetic plastic surgery.

7. The method of claim 1, wherein the wound comprises an internal organ.

8. The method of claim 7, wherein the wound is repaired in an endoscopic or other minimally invasive procedure.

9. The method of claim 1, wherein the wound is repaired in the field e.g. in military combat.

10. The method of claim 1, wherein the light comprises UV or visible light.

11. The method of claim 1, wherein a solvated primer solution is applied to the wound followed by application of a nonsolvated adhesive.

12. The method of claim 1, wherein the photopolymerizable adhesive is applied as a solvated adhesive blend.

13. The method of claim 4, wherein the wound comprises a traumatic wound.

14. The method of claim 1, wherein the adhesive is provided as a surgical kit comprising sterile single dose units that also contains at least one disposable device for application in addition to disposable wells for dispensing.

15. The method of claim 14, wherein the disposable device is selected from a microsponge, a syringe and a cannulae.
